(19) United States
(12) Patent Application Publication
(10) Pub. No.: US 2002/0103083 A1
(43) Pub. Date: Aug. 1, 2002

(54) Promoting deeper root development, reducing nitrogen fertilizer usage, imparting drought resistance, and increasing tolerance to adverse soil conditions in plants

(76) Inventor: Gary E. Harman, Geneva, NY (US)

Correspondence Address: Michael L. Goldman, Esq., NIXON PEABODY LLP, Clinton Square, P.O. Box 31051, Rochester, NY, 14603-1051, US

(21) Appl. No.: 09/927,966

(22) Filed: Aug. 10, 2001

Related U.S. Application Data
(60) Provisional application No. 60/224,572, filed on Aug. 11, 2000.

(51) Int. Cl. C12N 15/82; A01N 63/00

(52) U.S. Cl. 504/117

(57) ABSTRACT

The present invention relates to methods of promoting deeper root development, reducing nitrogen usage in plants, imparting drought resistance and increased tolerance of plants to adverse soil conditions, by the application of biologic plant root development agents to a plant or plant seed.

This application claims the benefit of U.S. Provisional Patent Application Ser. No. 60/224,572, filed Aug. 11, 2000.

### FIELD OF THE INVENTION

The present invention relates to methods of promoting deeper root development, reducing nitrogen fertilizer usage, enhancing plant growth, and imparting drought resistance and increased tolerance to adverse growing conditions by applying plant root development agents to seeds and plants.

### BACKGROUND OF THE INVENTION

Pollution of the environment by pesticides creates problems and concerns. Residues of pesticides may originate when a crop is treated with a chemical or exposed unintentionally by drift, in irrigation water, in feed, or other routes. In 1993, the National Research Council of the USA published a report indicating that pesticides pose unique risks to children (National Research Council, USA., “Pesticides in the Diets of Infants and Children,” Washington, D.C.: National Academy Press, 386 pg. (1993)). Consequently, regulatory agencies are examining the effects of many pesticides, requiring registrants to conduct acute, subchronic, and development neurotoxicity studies for at least 140 pesticides (Environmental Protection Agency, USA, “Implementing the Food Quality Protection Act. A Progress Report,” Washington, D.C.: 51 pg. (1999)). Pesticides heavily used in agriculture have been banned over the past several years by several governments. The USA recently banned sales of some uses of chlorpyrifos, a widely used insecticide, and has curtailed use of the fungicides chlorthalonil and iprodione. Methyl bromide, a widely used soil sterilizing agent (about 70,000 tons are used annually for this purpose) is a particularly vexing problem. In some locations, especially where winters are warm, this fumigant is necessary for efficient crop production. In its absence, diseases and pests can make crop production uneconomical. However, methyl bromide is damaging to the ozone layer as well as being toxic to humans in the vicinity of the application. The damage to the ozone layer is severe, and there is a worldwide ban that will be completed by 2005 in the developed countries and a few years later in the developing countries. (Environment Programme, U.N.: “Montreal Protocol on Substances that Deplete the Ozone Layer.” 375 pg (1998)).

Modern intensive agriculture creates other problems as well. Nitrogen fertilizer that is not taken up by crops pollutes waterways. In the USA, concentrations of nitrates at or above Environmental Protection Agency (“EPA”) maximum contaminant level (MCL) of 10 ppm for drinking water were detected in 15% of samples collected in shallow ground water beneath agricultural and urban lands, which is a concern for rural areas where shallow aquifers are used for drinking water supplies (U.S. Geological Survey, #6201 (1999)). Within the Mississippi River basin, nitrate may be found at concentrations approaching the MCL (Antweiler et al., “Nutrients in the Mississippi River,” U.S. Geological Survey Circular 1122:1-11(2000)). In the USA, rivers and wells often exceed the maximum EPA allowable nitrate level for drinking water of 10 ppm, especially in the spring when leaching from new maize plantings is at its peak. Non-point source nitrate pollution from farms contributes to the zone of hypoxia along the coast of the USA in the Gulf of Mexico and other regions, and may also encourage growth of toxic estuarine microbes such as Pfiesteria. These environmental costs are high—the EPA estimates that harmful algal blooms may have been responsible for an estimated $1,000,000,000 in economic losses during the past decade. Certainly not all of this loss can be attributed to agricultural activities; farmers, however, clearly will be required to bear increasingly large shares of these costs.

Various microorganisms long have been known to improve plant growth and productivity and reduce plant diseases (Burr et al., “Increased Potato Yields By Treatment of Seedpieces With Specific Strains of Pseudomonas fluorescens and P. putida,” Phytpathology 68:1377-1383 (1978); Schippers et al., “Plant Growth Control By Fluorescent Pseudomonads,” In: Chet, I. (ed.): Innovative Approaches to Plant Disease Control. John Wiley and Sons, New York, pp. 19-39 (1987); Weindling, “Trichoderma lignorum as a Parasite of Other Soil Fungi,” Phytpathology 22, 837-845 (1932)). However, only now are these organisms being used to alleviate environmental ills and improve productivity.

Many biocontrol agents control pests through direct activity against the pest. However, single microbial and other compounds have been discovered that mimic many of the beneficial effects of biocontrol agents, and the same strains appear to improve plant growth and productivity of plants. Understanding the mechanisms of these compounds and the organisms that produce them may provide methods to substantially improve plant agriculture through improved methods of plant growth and development with reduced reliance on potentially harmful chemicals.

The present invention is directed to overcoming these and other deficiencies in the art.

### SUMMARY OF THE INVENTION

The present invention relates to a method of promoting plant deep root development. This involves applying Trichoderma spp. to a plant or plant seed under conditions effective to achieve deeper roots in the soil in a treated plant or a plant grown from a treated seed than in untreated plants or plants grown from seed not treated with Trichoderma spp.

The present invention also relates to a method of reducing usage of nitrogen fertilizer in treating a plant. This involves applying a plant deep root developing agent to a plant or plant seed under conditions effective to reduce nitrogen fertilizer treatment of the plant. This method also involves achieving a level of plant growth like that achieved when treating the plant with the nitrogen fertilizer but not the plant deep root developing agent.

The present invention also relates to a method of imparting drought resistance to plants. This involves applying a plant deep root developing agent to a plant or plant seed under conditions effective to impart drought resistance to the plant or a plant grown from the plant seed.

The present invention also relates to a method of increasing tolerance of plants to adverse soil conditions. This involves applying a plant deep root developing agent to a plant or plant seed under conditions effective to impart resistance to adverse soil conditions of the plant. This method also involves achieving an improved level of plant growth in plants treated with the deep root developing agent.

The present invention provides a biologic alternative to the use of chemicals to enhance plant root development, plant growth, and crop yield. Such biologicals may be highly attractive to commercial agriculture in instances where the availability of chemical pesticides are lost to regulatory action or pest resistance and in which there are no adequate chemical replacements. They may also be used for replacement, or reduction of use, of agricultural chemicals in sensitive environments, in applications where biologicals accomplish tasks not possible for chemical pesticides; and where organic applications are preferred.

### BRIEF DESCRIPTION OF THE DRAWINGS

FIG. 1 shows the colonization of sweet corn root hairs following a seed treatment with the protoplast fusion progeny of T. harzianum 1295-22 (“T-22”) (ATCC 20847).

FIGS. 2A-B demonstrate replacement of endogenous fungi by application of T. virens strain 41.

FIG. 2A shows the percentage of total culturable fungi obtained from roots.

FIG. 2B shows the percentage of total culturable fungi obtained from soil.

FIGS. 3A-B show enhanced root development in field crops induced by T-22.

FIG. 3A shows roots of sweet corn grown from seeds treated with T-22™ Planter Box treatment (BioWorks, Inc., Geneva, N.Y.) (right) and no treatment (left).

FIG. 3B shows soybean plants with roots grown from seeds treated with T-22 (right) and no treatment (left).

FIGS. 4A-B show the relative growth of treated and untreated sweet corn.

FIG. 4A shows corn plants grown without T-22 treatment.

FIG. 4B shows corn plants whose roots were colonized by T-22 following treatment.

FIGS. 5A-C show enhanced deep rooting in field corn induced by T-22.

FIG. 5A shows the numbers of roots at different depths of mature (2 m-tall) field corn (‘E238’) in field trials with and without root colonization by T-22.

FIG. 5B shows the appearance of root intercepts of mature (2 m-tall) field corn (‘E238’) in field trials with (right) and without (left) root colonization by T-22, marked by map pins in single 25×25 cm squares 25 to 50 cm (top) or 50 to 75 cm (bottom) below the ground level.

FIG. 5C shows drought tolerance in corn induced by T-22. Corn without T-22 is shown on the right, T-22-treated corn is shown on the left.

FIGS. 6A-F show the interaction of T-22 and nitrogen fertilizer on corn growth and yield.

FIG. 6A shows corn growth response to different levels of nitrogen from T-22-treated and nontreated plots two weeks after side dressing.

FIG. 6B shows corn growth response to nitrogen four weeks after application of various levels of nitrogen.

FIG. 6C shows leaf greenness of corn at tasselling as a function of treatment with T-22 and nitrogen application level as measured with a Minolta SPAD 502 meter.

FIG. 6D shows stalk diameter of corn at harvest as influenced by treatment with T-22 and different levels of nitrogen.

FIG. 6E shows grain yield as influenced by treatment with T-22 and different levels of nitrogen.

FIG. 6F shows the yield of silage as influenced by the presence or absence of T-22 and level of nitrogen fertilizer.

### DETAILED DESCRIPTION OF THE INVENTION

The present invention relates to a method of promoting plant deep root development. This involves applying Trichoderma spp. to a plant or plant seed under conditions effective to achieve deeper roots in the soil in a treated plant or a plant grown from a treated seed than in untreated plants or plants grown from seed not treated with the Trichoderma spp. Trichoderma proliferates and colonizes the root system, producing deeper root growth and enhanced plant development. These benefits are probably the result of both displacement and control of deleterious root microflora and by direct effects on plants by as yet unidentified biochemicals. Exemplary organisms suitable for this aspect of the present invention are fungi in the genus Trichoderma (U.S. Pat. No. 5,260,213 to Harman et. al., which is hereby incorporated by reference in its entirety), including Trichoderma harzianum; the protoplast fusion progeny of Trichoderma harzianum 1295-22, known as “T-22”, (ATCC 20847) and T-22™ (BioWorks, Inc., Geneva, N.Y.); and T. virens, formerly classified as Gliocladium virens (U.S. Pat. No. 5,165,928 to Smith et al., which is hereby incorporated by reference in its entirety). Deep rooting is used herein to refer to any situation where there is a 10% or greater increase in root mass or root numbers at 4 cm and more below the soil surface in plants treated with a deep root enhancing agent of the present invention as compared with nontreated plants. Those skilled in the art will appreciate that the depth at which roots are measured will vary considerably; for example, turfgrass roots may extend only 4 cm below the soil surface, whereas corn roots may be as much as a meter deep.

The deep rooting agents of the present invention can be added to plant production systems in a number of ways, as follows.

Incorporation into Soils or Greenhouse Planting Mixes.

Beneficial microbes may be formulated or mixed to prepare granules, dusts or liquid suspensions. These can be mixed directly into soils or planting mixes. The preparations are then mixed into the soil or planting mix volume for greenhouse applications or into the upper volume of field soil (Harman, G. E., “The Dogmas and Myths of Biocontrol. Changes in Perceptions Based on Research with Trichoderma harzianum T-22,” Plant Dis. 84, 377-393 (2000), which is hereby incorporated by reference in its entirety). Equipment and procedures for such applications are well known and used in various agricultural industries. Typical rates are 0.2 to 10 kg of product containing 10⁷ to 10⁹ colony forming units (cfu) per cubic meter of planting mix or soil.

Drenches for Greenhouse or Nursery Soils and Soil Mixes.

Liquid suspensions of the beneficial microorganisms can be prepared by mixing dry powder formulations into water or other aqueous carrier, including fertilizer solutions, or by diluting a liquid formulation containing the microbe in water or other aqueous solutions, including those containing fertilizers. Such solutions can then be used to water planting mixes either prior to planting or else when plants are actively growing. Typically 10 to 400 ml of product (typically 150 μm or smaller in particle size) containing 10⁷ to 10⁹ cfu are mixed with 100 L of water for such applications.

Slurry Film-coated or Pelleted Seeds.

Seeds are commonly treated using slurry, film-coating or pelleting by processes well known in the trade (Harman et al., “Factors Affecting Trichoderma hamatum Applied to Seeds As a Biocontrol Agent,” Phytopathology 71: 569-572 (1981); Taylor et al., “Concepts and Technologies of Selected Seed Treatments,” Ann. Rev. Phytopathol. 28: 321-339 (1990), which is hereby incorporated by reference in its entirety). The beneficial microbial agents of the present invention can effectively be added to any such treatment, providing that the formulations do not contain materials injurious to the beneficial organism. Depending on the microbe in question, this may include chemical fungicides. Typically, powder or liquid formulations (10⁷ to 10¹⁰ cfu/g) of the organism are suspended in aqueous suspensions to give a bioactive level of the microbe. The liquid typically contains adhesives and other materials to provide a good level of coverage of the seeds and may also improve its shape for planting or its cosmetic appeal.

Dust or Planter Box Treatments for Roots, Bulbs and Seeds.

Dry powders containing beneficial microbes can be applied as a dust to roots, bulbs or seeds. Generally fine powders (usually 250 μm or smaller) are dusted onto seeds, bulbs or roots to the maximum carrying powder (i.e., until no more powder will adhere to the treated surface). Such powders typically contain 10⁷ to 10⁹ cfu/g.

In-furrow Application.

Liquid suspensions of products may be prepared as described above for preparing drenches. Such materials may be added to the furrow into which seeds are planted or small plants are transplanted. Equipment for such applications are widely used in the agricultural industry. Typical rates of application are 0.5 to 10 kg of product (10⁷ to 10⁹ cfu/g) per hectare of field.

Broadcast Applications.

Granules, as described above, can be broadcast onto soil surfaces that contain growing plants, to soil at the time of planting, or onto soils into which seeds or plants will be planted. Typical rates range from I to 500 kg of product containing 10⁷ to 10⁹ cfu/g depending upon the plants to be treated and the goals of the treatment. Alternatively, spray solutions can be prepared as described above, and applied to give similar rates (Harman, G. E., “The Dogmas and Myths of Biocontrol. Changes in Perceptions Based on Research with Trichoderma harzianum T-22,” Plant Dis. 84, 377-393 (2000); Lo et al., “Biological Control of Turfgrass Diseases With a Rhizosphere Competent Strain of Trichoderma harzianum,” Plant Dis. 80, 736-741(1996); Lo et al., “Improved Biocontrol Efficacy of Trichoderma harzianum 1295-22 For Foliar Phases of Turf Diseases By Use of Spray Applications,” Plant Dis. 81: 1132-1138 (1997), which are hereby incorporated by reference in their entirety).

For the purposes of the present invention, all treatments are designed to accomplish the same purpose, i.e., to provide a means of application that will result in effective colonization of the root by the beneficial microbe (Harman and Björkman, “Potential and Existing Uses of Trichoderma and Gliocladium For Plant Disease Control and Plant Growth Enhancement,” In: Harman, G. E. and Kubicek, C. P. (ed.): Trichoderma and Gliocladium, Vol. 2. Taylor and Francis, London, pp. 229-265 (1998), which is hereby incorporated by reference in its entirety).

The methods of the present invention can be utilized to treat a wide variety of plants or their seeds. Suitable plants include dicots and monocots. More particularly, useful crop plants can include: alfalfa, rice, wheat, barley, rye, cotton, sunflower, peanut, corn, potato, sweet potato, bean, pea, chicory, lettuce, endive, cabbage, brussel sprout, beet, parsnip, turnip, cauliflower, broccoli, radish, spinach, onion, garlic, eggplant, pepper, celery, carrot, squash, pumpkin, zucchini, cucumber, apple, pear, melon, citrus, strawberry, grape, raspberry, pineapple, soybean, tobacco, tomato, sorghum, and sugarcane. Examples of suitable ornamental plants are: Arabidopsis thaliana, Saintpaulia, petunia, pelargonium, poinsettia, chrysanthemum, carnation, zinnia, and turfgrasses.

The present invention also relates to a method of reducing usage of nitrogen fertilizer in treating a plant. This involves applying one or more plant deep root developing agents of the present invention to a plant or plant seed under conditions effective to reduce nitrogen fertilizer treatment of the plant, while achieving a level of plant growth like that achieved when treating the plant with the nitrogen fertilizer but not the plant deep root developing agent. This aspect of the present invention may be accomplished by applying a plant deep root developing agent to the seeds of a plant. Seed treatment may be carried out by treating the seed as described above. Alternatively, an effective amount of one of more root enhancing agents can be added directly to the soil, for example, as spores to the soil volume, as in-furrow or greenhouse soil drenches, or as broadcast granules on the surface of planted soil. The root enhancing agent of the present invention may also be applied as incorporated granules in greenhouse planting mixes, or as a conidial suspension in greenhouse potting mixes. Regardless of the mode of application, plants treated with one or more of the deep root developing agents of the present invention show deeper rooting, with plant growth and crop yield comparable to, or better than, plants not treated with a deep root developing agent in accordance with the present invention, but that have been treated with commercially available nitrogen fertilizers.

The deep root developing agents of the present invention are organisms with strong abilities to colonize roots. This ability is known as rhizosphere competence, which is used herein to describe those organisms capable of colonizing the root surface or the surface plus surrounding soil volume (rhizoplane and rhizosphere, respectively), when applied as a seed or other point source at the time of planting in absence of bulk flow of water. Thus, the agents of the present invention have the physiological and genetic ability to proliferate the root as its develops. Rhizosphere competence is not an absolute term, and degrees of this ability may occur among strains (Harman, G. E., “The Development and Benefits of Rhizosphere Competent Fungi for Biological Control of Plant Pathogens,” J. Plant Nutrition 15:835-843 (1992); U.S. Pat. Nos. 4,996,157 and 5,165,928 to Smith, which are hereby incorporated by reference in their entirety). Other organisms, including those in the genera Bacillus, Pseudomonas and Burkholderia also possess good root competence (Brannen et al., “Kodiak: A Successful Biological-Control Product for Suppression of Soil-Borne Plant Pathogens of Cotton,” J. Industr. Microbiol. Biotechnol. 19 (1997) 169-171 (1997); Kloepper et al. “Plant Growth Promoting Rhizobacteria As Inducers of Systemic Acquired Resistance,” In: Lumsden, R. D. and Vaughn, J. L. (ed.): Pest Management: Biologically Based Technologies. Washington, D.C., pp. 10-20 (1993), which are hereby incorporated by reference in their entirety). Procedures for measuring rhizosphere competence are known to the skilled in the art (Harman et al., “Combining Effective Strains of Trichoderma harzianum and Solid Matrix Priming to Improve Biological Seed Treatments,” Plant Dis. 73:631-637 (1989); Harman, G. E., “The Dogmas and Myths of Biocontrol. Changes in Perceptions Based on Research with Trichoderma harzianum T-22,” Plant Dis. 84, 377-393 (2000); Kloepper et al., “A Review Of Issues Related To Measuring Colonization Of Plant Roots By Bacteria,” Can J. Microbiol. 38, 1219-1232 (1992), which are hereby incorporated by reference in their entirety).

Either fungal or bacterial agents may be rhizosphere competent. Examples of organisms with these capabilities which are suitable as root development enhancing agents of the present invention are beneficial microorganisms including, but not limited to, fungi in the genus Trichoderma (U.S. Pat. No. 5,260,213 to Harman et. al., which is hereby incorporated by reference in its entirety), including T. virens, formerly classified as Gliocladium virens (U.S. Pat. No. 5,165,928, to Smith et al., which is hereby incorporated by reference in its entirety); and bacteria in the genus Bacillus (Raupauch-Georg et al., “Mixtures of Plant Growth-Promoting Rhizobacteria Enhance Biological Control of Multiple Cucumber Pathogens,” Phytpathology 88 (1158-1164) (1998), which is hereby incorporated by reference in its entirety); Pseudomonas and Burkholderia (Burr et al., “Increased Potato Yields by Treatment of Seedpieces with Specific Strains of Pseudomonas fluorescens and P. putida,” Phytpathology 68 (1377-1383) (1978), which is hereby incorporated by reference in its entirety); Streptomyces, and Fusarium. The biological deep root developing agents of the present invention can be produced in large quantities in either liquid or semi-solid fermentation by routine microbial techniques, such as those described in Harman et al., “Potential and Existing Uses of Trichoderma and Gliocladium For Plant Disease Control and Plant Growth Enhancement,” In: Harman, G. E. and Kubicek, C. P. (ed.): Trichoderma and Gliocladium, Vol. 2. Taylor and Francis, London (1998), which is hereby incorporated by reference in its entirety. Those skilled in the art will appreciate that the physiology and type of propagule (e.g., hyphae, conidia, or chlamydospores) of the source organism will dictate preparation schema and optimization of yield.

In one aspect of the present invention, the deep root developing agent is highly rhizosphere competent fused strain of T. harzianum known as “T-22” (ATCC 20847) (U.S. Pat. No. 5,260,213 to Harman et al.; Harman, G. E., “The Dogmas and Myths of Biocontrol. Changes in Perceptions Based on Research with Trichoderma harzianum T-22,” Plant Dis. 84, 377-393 (2000), which are hereby incorporated by reference in their entirety). Any natural, mutant or fused strains of the genera of the present invention shown to be rhizosphere competent are also suitable for all aspects of the present invention.

Also suitable as a root enhancing agent of the present invention is humic acid. Humic acid is defined herein as any organic acid that is derived from humus (Academic Press Dictionary of Science and Technology, Academic Press, New York, 2432 pg. (1992), which is hereby incorporated by reference in its entirety). Humus refers to the relatively inert organic material resulting from decomposition of plant and animal matter. Humic acid may be used alone or in combination with one or more species of beneficial microorganisms.

A conservative expectation is that seed treatment with T. harzianum protoplast fusion progeny T-22 (ATCC 20847), for example, can reduce nitrogen fertilizer usage in maize by at least 25% in commercial agriculture with no yield penalty and can reduce nitrate leaching even more. At current (high) nitrogen fertilizer costs, the net savings to U.S. maize growers will be $50-100 million annually at a 10-20% national adoption rate. The genetic and biochemical bases for this increased ability of maize to utilize nitrogen fertilizer in the presence of T-22 are unknown, but occurs reliably at low soil nitrogen levels whenever roots are colonized. Moreover, root development is enhanced as much as a meter below the soil surface. This enhancement is not limited to maize but occurs on many crops. This technology has potential not only for countries such as the USA but also in developing countries where only low inputs of nitrogen fertilizer are affordable.

Another aspect of the present invention is a method of imparting drought resistance to plants. This involves applying a plant deep root developing agent to a plant or plant seed under conditions effective to impart drought resistance to the plant or a plant grown from the plant seed.

This aspect of the present invention can be accomplished by seed treatment and direct or indirect treatment of plants, all as described above, with one or more of the deep root developing agents of the present invention. This aspect of the present invention appears to be one of the substantial and unexpected advantages to plant growth and productivity that can be conferred by strongly rhizosphere competent biological control agents. Field trials, discussed in greater detail in the Examples below, were conducted to ascertain the depth and degree of root enhancement conferred by treatment with the root enhancing agents of the present invention. In particular, these studies showed a significant increase in root intercepts, i.e., root density, with, versus without, T-22 root colonization. This greater root density can bestow substantial benefits to crops, especially in dry growing seasons, thereby reducing the sensitivity of crops to drought stress.

The present invention also relates to a method of increasing the tolerance of plants to adverse soil conditions, such as highly compacted soils. This involves applying one or more of the plant deep root developing agents of the present invention to a plant or plant seed under conditions effective to impart resistance to adverse soil conditions of the plant. Application of the deep root developing agents may be by seed treatment or direct application to roots or soil as described above. This aspect of the present invention also involves achieving an improved level of plant growth for plants treated with the plant deep root developing agents of the present invention, in comparison with plants grown in adverse soil conditions which are not treated according to the present invention.

### EXAMPLES

### Example 1

### Hyphal Colonization of Root by T. harzianum

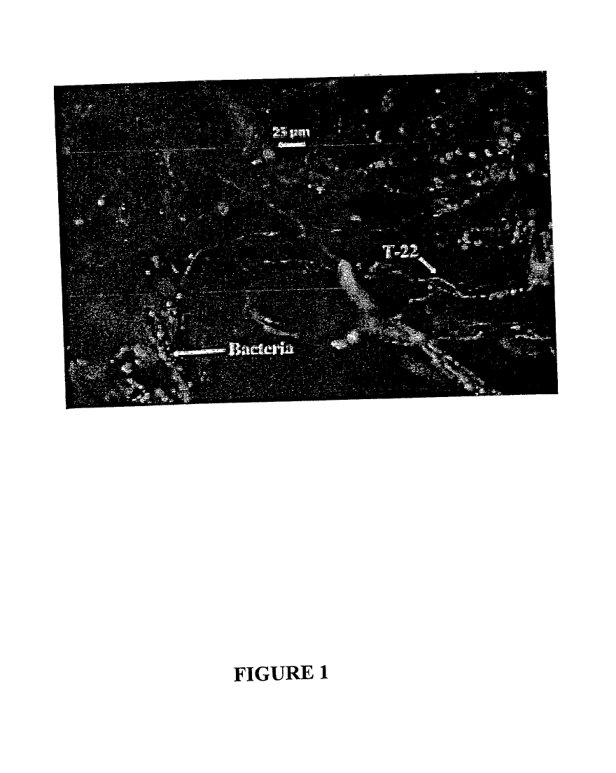

FIG. 1 shows the colonization of sweet corn root hairs following seed treatment with T. harzianum strain T-22. Root samples from plants grown in greenhouse potting soil were removed and washed. The root surfaces were oxidized in 1% periodic acid, rinsed and counterstained for 20 sec in Schiff's Reagent. They were then rinsed and mounted in 0.1 μg/μL 4′,6 diamidino-2-phenylindole (DAPI, Sigma, St. Louis, Mo.) to stain DNA of the inhabiting microbes and observed using epifluorescent microscopy. The fibrillar network of T-22 as well as bacterial colonies are indicated in FIG. 1. Roots of similar plants grown in the absence of T-22 lacked this hyphal network.

These results demonstrate that T-22 not only colonized roots but that other microflora were displaced, thereby changing the root microfloral composition. Moreover, there was almost no effect of soil type or geographic location on this ability. An exception to this generalization occurred on cotton roots at the height of the summer from fields outside Phoenix, Ariz. T-22 was present only sporadically on roots obtained from these hot, dry soils. In other studies, T-22 has provided advantages to cotton. Importantly, T-22 has equal ability to colonize roots in both alkaline and acidic soils (Lo et al., “Biological Control of Turfgrass Diseases With a Rhizosphere Competent Strain of Trichoderma harzianum,” Plant Dis. 80, 736-741(1996), which is hereby incorporated by reference in its entirety) and across soil types ranging from sandy to heavy and with a wide variation of organic matter content (Harman, G. E., and Björkman, “Potential And Existing Uses of Trichoderma and Gliocladium For Plant Disease Control and Plant Growth Enhancement,” pp. 229-265 in: Trichoderma and Gliocladium, vol. 2. G. E. Harman and C. P. Kubicek, eds. Taylor and Francis, London (1998), which is hereby incorporated by reference in its entirety), and various levels of compaction. Further, the method of application was not important. Colonization was obtained over the entire root surface when T-22 was added as a seed treatment, as broadcast granules on the surface of planted soil, as an in-furrow granule or drench, as incorporated granules in greenhouse planting mixes or as a conidial suspension in greenhouse potting mixes. T-22 grew onto all newly formed root surfaces.

### Example 2

### Colonization Results in Replacement of Endogenous Fungi in Roots and Soil

T. virens strain 41 was obtained from roots of pea plants in an Aphanomyces-suppressive soil and found capable of controlling Phytophthora spp. and other pathogenic fungi (Smith et al., “Potential For Biological Control of Phytophthora Root And Crown Rots Of Apple By Trichoderma and Gliocladium spp.,” Phytpathology 80:880-885 (1990), which is hereby incorporated by reference in its entirety). A three-year field study was conducted on the control of Phytophthora on raspberry. Raised beds, metalaxyl treatments, metalaxyl+T. virens and T. virens added twice a year or once a month during the growing season, were evaluated in the study.

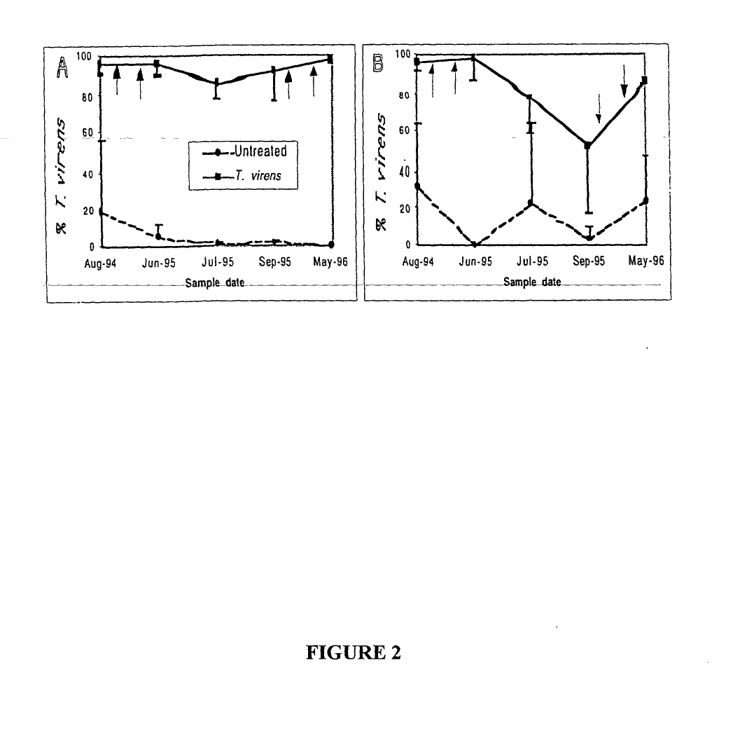

T. virens strain 41 was applied as a granular application (62 kg/ha) in a strip 1 m wide on rows of raspberries once in the spring and once the fall. The initial application was at the time of planting and thereafter granules were banded on the soil surface. Two soil cores were taken from each plot and dilution plated onto acidified potato dextrose and amended with Igepal Co-630 as a colony restrictor (Taylor et al, “Liquid Coating Formulation For the Application of Biological Seed Treatments of Trichoderma harzianum,” Biol. Control 1:16-22 (1991), which is hereby incorporated by reference in its entirety). Fungi other than Trichoderma were easily distinguished on the basis of morphology. T. virens was easily be separated from other Trichoderma strains on the acid PDA medium. On this medium, T. virens has a pure white coloration on its lower surface while native Trichoderma spp. were tan to brown. Root and soil fungal populations were assayed at monthly intervals over the course of the season. In the absence of application of strain 41, T. virens made up only a minor part of the total fungal population. However, when the biocontrol agent was added in any of the regimes noted above (Trichoderma spp. generally are resistant to metalaxyl), the T. virens populations became established and persisted as the dominant fungus colonizing roots and soil over the entire three growing seasons of the experiment. FIGS. 2A-B demonstrate replacement of endogenous fungi by application of T. virens strain 41. The percentage of total culturable fungi obtained from roots is shown in FIG. 2A. Shown in FIG. 2B is the percentage of total culturable fungi obtained from soil. Data shown are from plots of cv. ‘Newburgh’ raspberries on raised beds in a field soil heavily infested with Phytophthora fragariae var. rubri. Arrows indicate application times. Each treatment was replicated four times and error bars represent standard deviations.

T. virens was present in similar numbers over the entire growing season, became the dominant culturable fungus in the soil, and persisted extremely well. The behavior of strain 41 differs from that of T-22 in one very important respect: T-22 does not become dominant in the soil but only on the roots. Conversely, strain 41 establishes dominance in both habitats.

In the raspberry study, one of the organisms that T. virens was displacing was T. harzianum. On roots of turfgrass, however, T. virens is the most commonly isolated native microfloral component and makes up the majority of the isolates obtained. If, however, T-22 granules were applied either at high rates or repeatedly, T-22 displaced T. virens and other fungi and became the most numerous organism, frequently making up more than 50% of the total fungi isolated from the root zone.

### Example 3

### Promotion of Deep Rooting

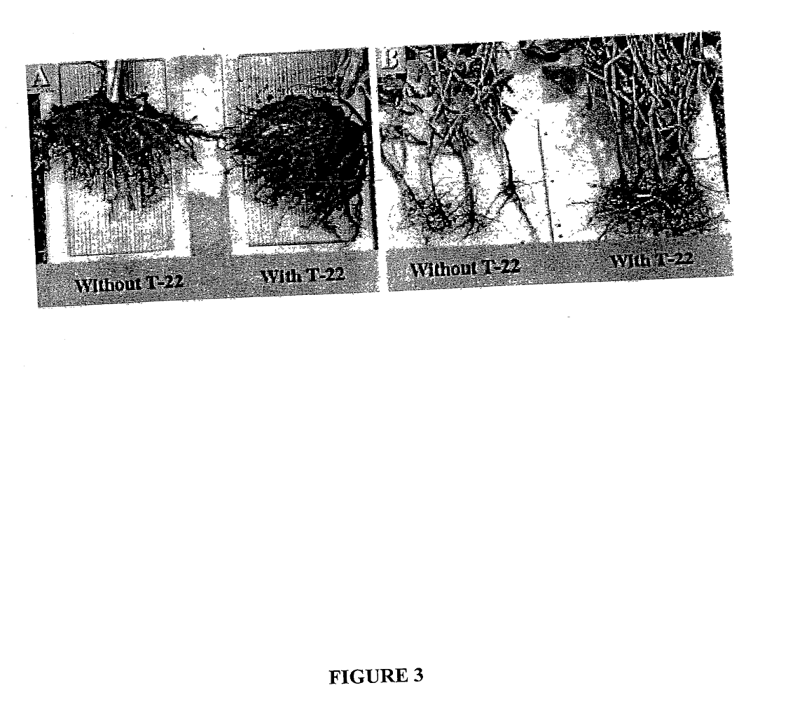

The consequences of root colonization by Trichoderma spp. can be profound in terms of plant disease control and plant growth and productivity (Baker et al., “The Controlled Experiment In The Scientific Method With Special Emphasis On Biological Control,” Phytpathology 74:1019-1021 (1984); Chang at al., “Increased Growth Of Plants In The Presence Of The Biological Control Agent Trichoderma harzianum.” Plant Dis. 70:145-148 (1986), which are hereby incorporated by reference in their entirety). Not only can colonized roots be protected against disease by T-22 but they frequently are larger and more robust. A typical result of deeper root development in field crops is shown in FIGS. 3A-B. FIG. 3A shows roots of sweet corn grown from seeds with (right) and without (left) T-22™ Planter Box (BioWorks, Inc., Geneva, N.Y.) treatment. Although roots colonized by T-22 were more abundant, in this particular trial yields were similar regardless of treatment; conditions of growth were good and no yield advantage was provided by the improved root system. FIG. 3B shows soybean plants with roots grown from seed with (right) and without (left) T-22 treatment. This increased root development may occur as a consequence of control of clinical or subclinical pathogens as suggested several years ago for Pseudomonas spp. (Burr et al., “Increased Potato Yields by Treatment of Seedpieces with Specific Strains of Pseudomonas fluorescens and P. putida,” Phytpathology 68 (1377-1383) (1978), which is hereby incorporated by reference in its entirety). However, T-22 may have direct effects upon plant metabolism as well. In limited studies, it was observed that T-22 was as effective as a commercial rooting hormone in inducing rooting of tomato cuttings, although callus tissue was not formed on the base of the cuttings as occurs with commercial hormone preparations. See Table 1. Further, T95, which was one of the parental strains used in the fusion that gave rise to T-22, increased plant growth even under axenic conditions (Windham et al., “A Mechanism For Increased Plant Growth Induced By Trichoderma spp.,” Phytopath. 76:518-521 (1986), which is hereby incorporated by reference in its entirety). Also, cucumber plants grown in axenic hydroponic conditions were larger in the presence of T. harzianum strain T-203 than in its absence (Yedidia et al., “Induction Of Defense Responses In Cucumber Plants (Cucumis sativus L.) By The Biocontrol Agent Trichoderma harzianum,” Appl. Environ. Microbiol. 65:1061-1070 (1999), which is hereby incorporated by reference in its entirety).

TABLE 1
Enhancement of rooting and root elongation of
cuttings of a Solanum x Lycopersicon hybrid as
a consequence of dipping of stem cuttings in root
hormone treatment powder (1% indole butryic acid) or
in T-22 drench.

| Treatment | Numbers of Roots^{a}_{_{—}} | Length of Roots (mm) |
|---|---|---|
| None | 1.2 ± 0.8 | 1.8 ± 0.6 |
| Root Hormone | 1.1 ± 0.3 | 1.6 ± 0.6 |
| T-22 | 2.5 ± 1.0 | 2.4 ± 0.6 |

^{a}Thirteen days after treatment just as roots were being initiated.

### Example 4

### Persistence of T. harzianum Root Colonization Provides Long Term Root Growth Enhancement

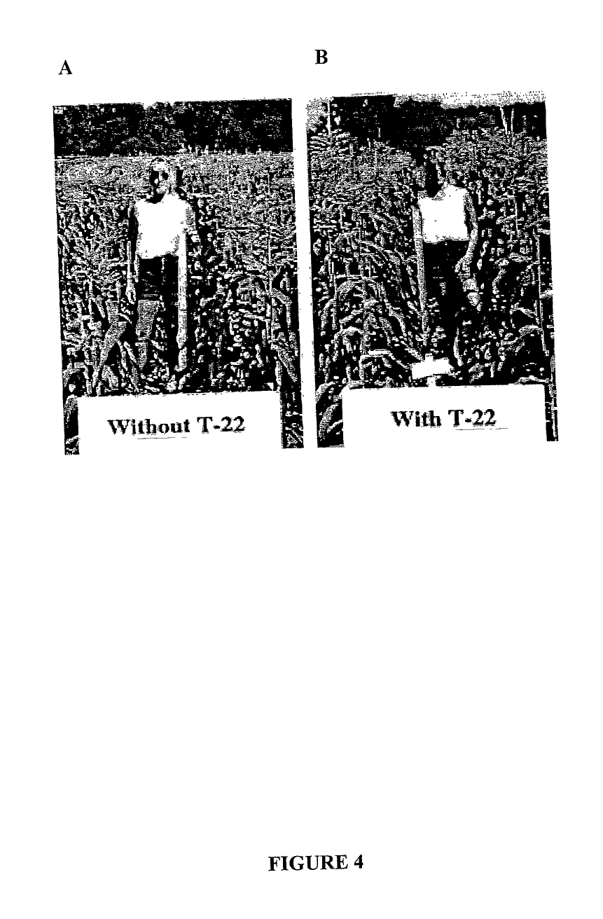

The length of time that T. harzianum strain T-22 can persist and proliferate on roots is considerable, as was demonstrated by the following experiment. A granular formulation of T-22 was applied by broadcast (about 10 kg/ha) at the same time that a rye grain cover crop was seeded in the fall. Control plots were seeded to the rye cover crop without T-22. Early the next spring, the roots were sampled and a high population of T-22 (around 10⁵ cfu/g dry weight of roots) was found on the roots where T-22 was broadcast but not on the control areas. In the following spring, the roots of the rye were found to be colonized by T-22. The small grain rye plants were killed by spraying with glyphosate. Sweet corn (‘Jubilee Supersweet’ with standard fungicide seed treatments) was planted into the field without tillage. Differences in growth of the corn between the treated and nontreated plots were evident over the entire season and were remarkably different at the end of the season. FIGS. 4A-B show this result. Corn grown on the field corn grown without T-22 is shown in FIG. 4A, and corn grown with earlier T-22 treatment is shown in FIG. 4B. As would be expected from the size of the plants, the yield of corn was also different, with 1.7 times more weight of ears being harvested from the T-22-treated plots versus those originally planted to the rye cover crops without T-22.

### Example 5

### Cover Cropping

Another example demonstrates long term effects of T-22. Plots were planted in the fall to T-22 treated or nontreated seed of a sorghum-sudan hybrid on a highly organic (muck) soil. The roots of the plants were sampled after the crop became established and there was one order of magnitude higher populations of Trichoderma spp. on plants grown from treated seeds than from nontreated seeds. The next spring onions were planted on either the sorghum+T-22 or sorghum without T-22 plots. Yields were greater in the presence of T-22 than in its absence. The best yields occurred in rows that also received a maneb+ridomyl drench to control early season diseases. With the integrated T-22+fungicide program, yields were increased 10% relative to the same program without T-22, which was highly significant both economically and statistically.

All of these examples demonstrate that, with T-22 and T. virens strain 41 at least, it is possible to achieve much more than transitory localized dominance of the rhizosphere, and in only some soils and seasons, as has been predicted for biocontrol agents. (Deacon, J. W., “Rhizosphere Constraints Affecting Biocontrol Organisms Applied To Seeds,” pp. 315-326 in: Seed Treatment: Progress and Prospects. T. Martin, ed. British Crop Protection Council, Farnham, UK. (1994), which is hereby incorporated by reference in its entirety). The results indicate that rhizosphere competence is real and can result in long-term root colonization, which in turn can provide quantifiable improvements in plant performance.

### Example 6

### Drought Resistance Acquired by Increased Deeper Roots

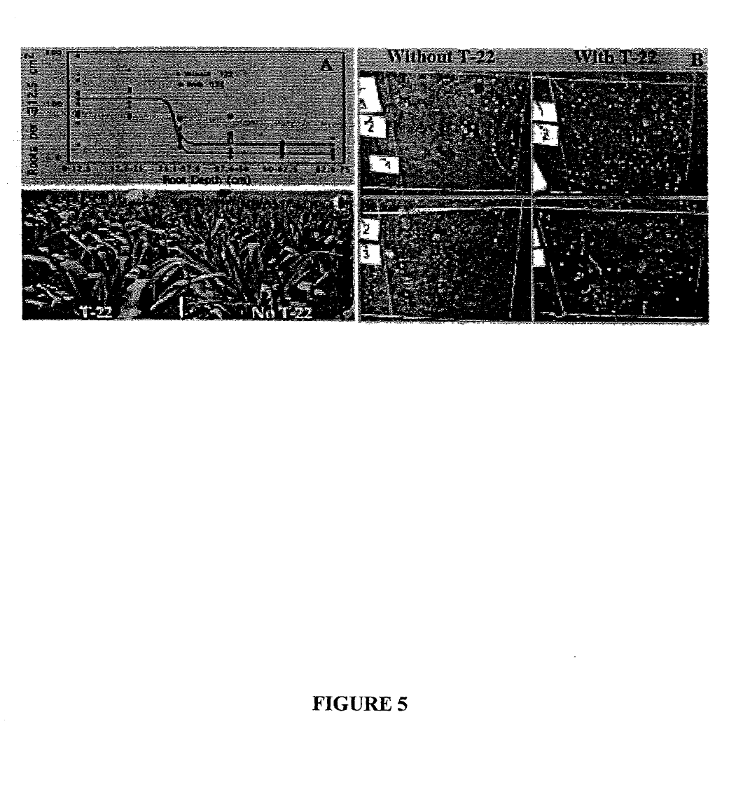

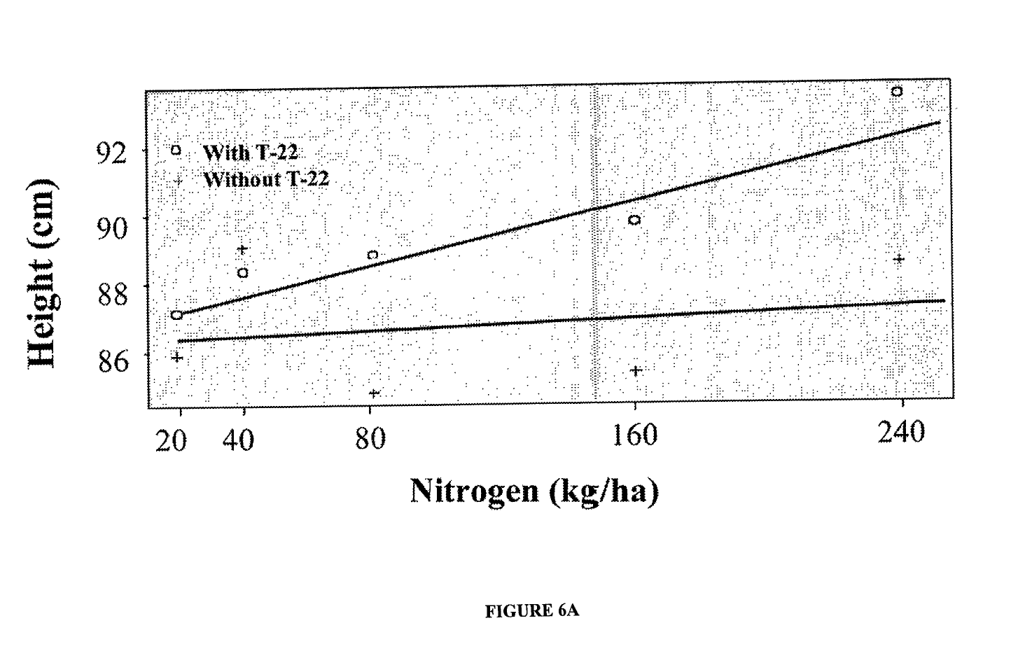

Trials with field corn were conducted in a sandy loam grower field to determine the depth and degree of root enhancement conferred by T-22 treatment. The field was planted to corn either treated (seed dusted), or not treated, with the commercial formulation of T-22 (BioWorks, Inc., Geneva, N.Y.) in alternating bands six rows wide throughout the field. At about the time of tasselling (about 60 days after planting), trenches were dug about 3.2 meters deep with a back hoe about 15 cm in front of rows of corn about 2 m tall. The exposed soil profile was washed with a power washer to reveal the roots. Grids 25×25 cm were established across and down the soil profile. Each root intercept was marked with a map pin with a differently colored head (different colors for each adjacent grid), the grids were individually photographed and the pins in each grid were removed, placed in separate boxes, and counted. Numbers of root intercepts were similar in corn with and without T-22 in the upper 25 cm of soil. However, roughly twice as many intercepts were found in the second and third grids when T-22 was present than when it was absent. FIG. 5A shows a graph of the resulting data. The data were modeled by logistic curves (76/72) of the form:

Numbers of roots=a+c/l^{-b(depth-m)}

where a=lower asymptote, a+c=the upper asymptote, m=the point of inflexion, and b=the slope parameter. The lines are significantly different at P=0.006. Data for the lower four regions of soil were fitted to linear regressions; there was no significant difference in slopes but the elevations were different at P=0.001 (graph not shown). This greater deep root density may be particularly beneficial to corn and other crops in dry growing seasons.

FIG. 5B shows the appearance of the field corn root intercepts graphically depicted in FIG. 5A, with (right) and without (left) root colonization by T-22, marked by map pins in single 25×25 cm squares 25 to 50 cm (top) or 50 to 75 cm (bottom) below the ground level. Squares shown were chosen because they approximate the average values for each treatment.

FIG. 5C shows drought tolerance in corn induced by T-22 in field trails which was probably the result of enhanced deep rooting. T-22 was applied as a dust to the seed according to manufacturer's recommendations (6-8 ounces per 100 lb. of seed; 170-225 g. per 45 kg seed) (BioWorks, Inc., Geneva, N.Y.). Corn without T-22 treatment (right) exhibited the typical leaf curl to reduce transpiration symptomatic of drought stress, while leaves of T-22-treated corn (left) did not.

### Example 7

### The Interaction of T-22 and Nitrogen Fertilizer on Corn Growth and Yield

It was noted that corn plants that grew from seeds treated with T-22 were greener than plants without T-22. Increased greenness in corn frequently is associated with higher levels of nitrogen uptake (Piekielek et al., “Use Of A Chlorophyll Meter To Predict Sidedress Nitrogen Requirements For Maize,” Agron. J. 84:59-65 (1992), which is hereby incorporated by reference in its entirety). It was considered that one of the effects of T-22 on colonized roots was increased efficacy of use of applied fertilizers, especially nitrogen. This was tested in a trial established with field corn in a commercial grower's field on a sandy loam at various levels of nitrogen fertility. Such treatments have been used for other studies in order to determine nitrogen efficiency as imposed by various treatments using negative exponential models (Klausner et al., “An Approach For Estimating A Decay Series For Organic Nitrogen In Animal Manure,” Agron. J. 86:897-903 (1994), which is hereby incorporated by reference in its entirety). The entire field was planted in bands six rows wide with seed treated with T-22 planter box alternating with six rows without the biocontrol agent. It was expected that the initial nitrate level would be low since the field had not received recent manure applications and because corn had been grown in the previous season. Pre-side dress nitrate soil tests (PSNT) verified that the endogenous nitrogen was indeed low, about 20 kg/ha. Nitrogen, in the form of ammonium nitrate, was banded and incorporated beside rows of corn at about the four-leaf stage to give a total, including the 20 kg/ha of residual nitrogen, of 20, 40, 80, 160, and 240 kg of total nitrogen per ha.

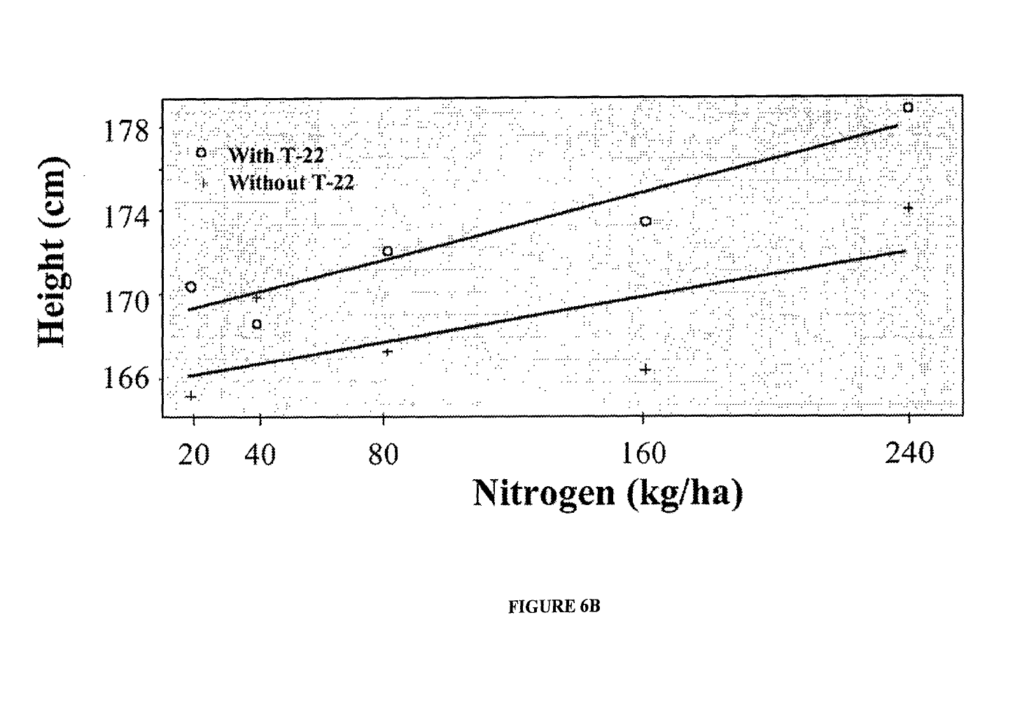

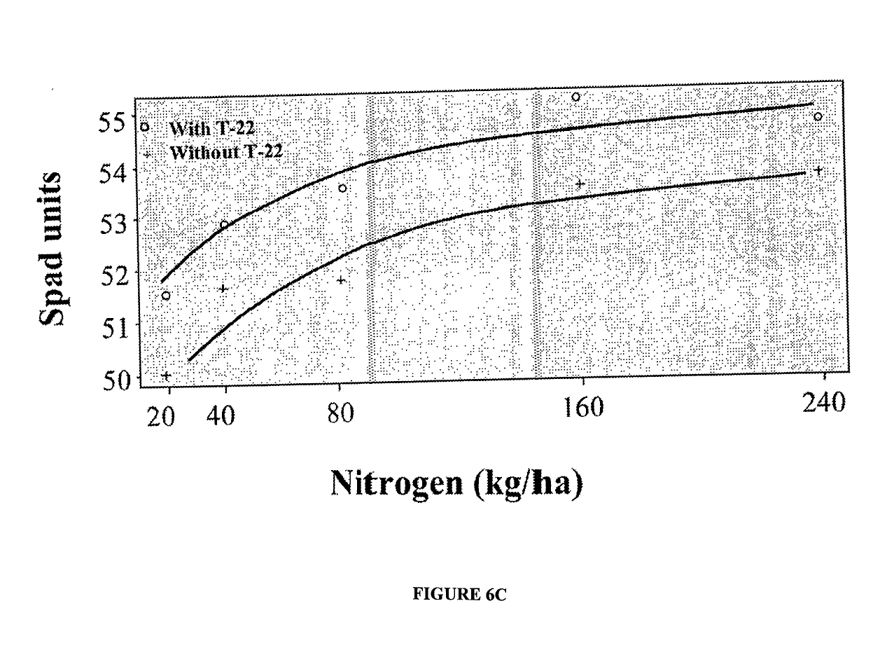

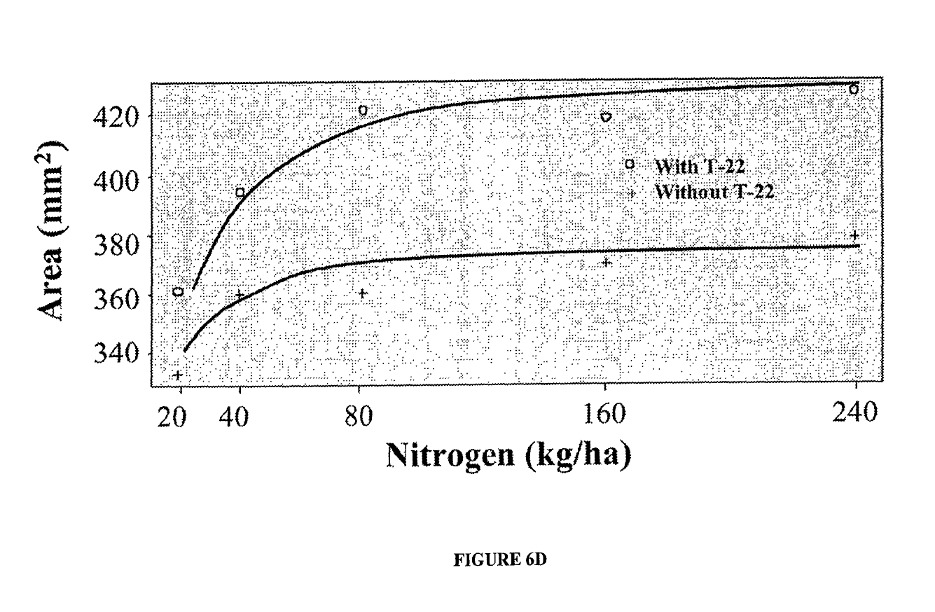

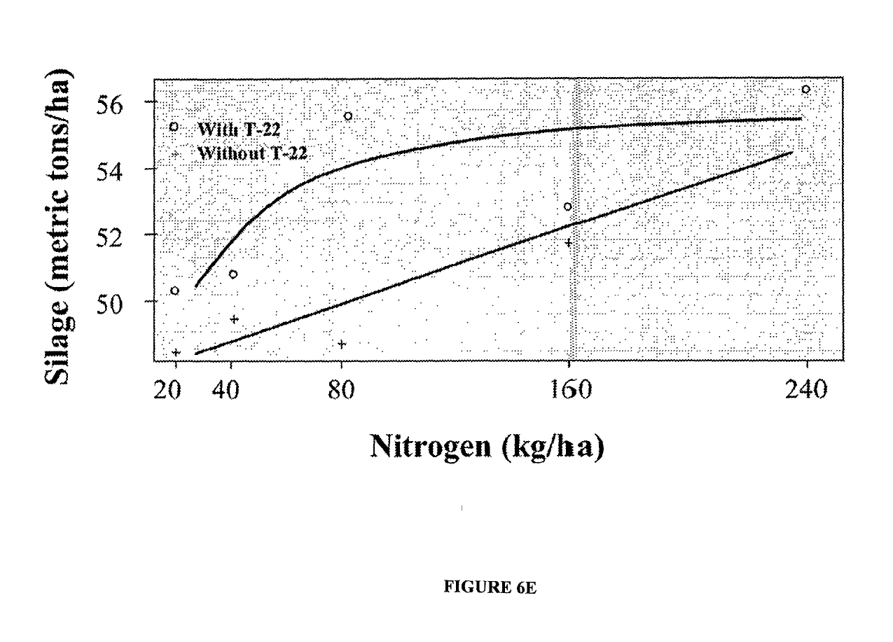

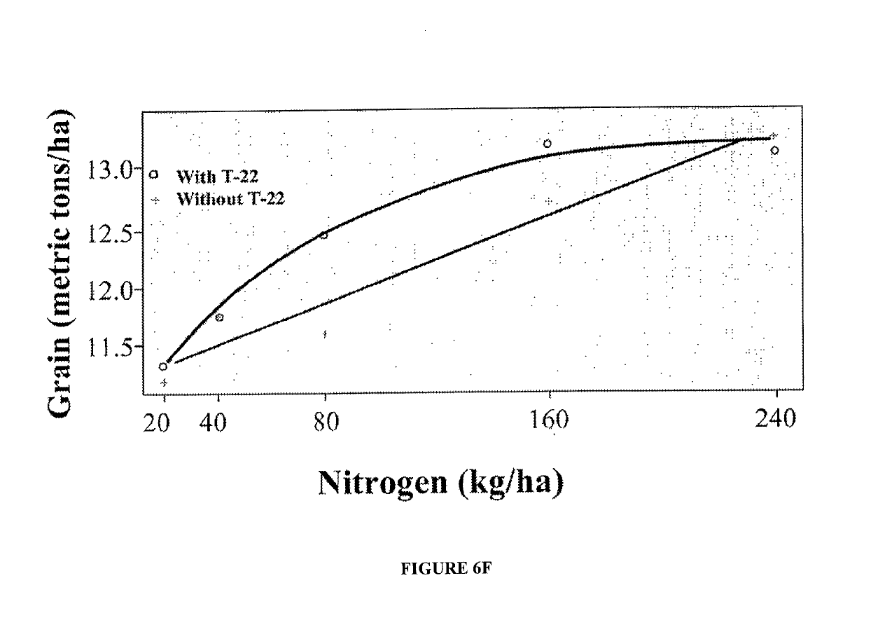

Differences were observed almost immediately. Heights of corn were measured two weeks after side dressing and those with T-22 responded more rapidly and remained larger for most of the growing season than control plots, as shown in FIG. 6A. Plants whose roots were colonized with T-22 responded to nitrogen, while those without T-22 did not. Both position and slope were significantly different based on regression analysis (P=0.001 and 0.04, respectively). FIG. 6B shows corn growth response to nitrogen four weeks after application of various levels of nitrogen. At this time, slopes were similar but the intercept of the curve was significantly different (P=0.001). At two and four weeks after nitrogen application there was no difference in corn greenness as determined by readings with a SPAD meter (Piekielek et al., “Use Of A Chlorophyll Meter To Predict Sidedress Nitrogen Requirements For Maize,” Agron. J. 84:59-65 (1992), which is hereby incorporated by reference in its entirety). However, later, at the time of tasselling, the T-22-treated plants were greener in a nitrogen dose-dependent manner, as shown in FIG. 6C. At tasselling, all plants were of similar heights but leaf greenness was significantly different (slope parameters were not significantly different but position was significantly different based on regression analyses at P=0.002). At maturity, there was a difference in stem diameter and grain and silage yields. FIG. 6D shows stalk diameter of corn at harvest as influenced by treatment with T-22 and different levels of nitrogen. Slope parameters were similar but the positions of the lines were significantly different at P=<0.001.

FIG. 6E shows yield of grain from corn plants as influenced by treatment with T-22 and different levels of nitrogen. Plants were grown with T-22 treatment, and 80 lbs of nitrogen fertilizer, or without T-22 treatment and 150 lbs nitrogen fertilizer. Yield was not significantly different, but treated plants used 53% less nitrogen.

The yield of silage as influenced by the presence or absence of T-22 and level of nitrogen fertilizer is shown in FIG. 6F. Perhaps the most important yield comparisons were the points at which nitrogen no longer gave a yield increase, presumably because the plants had as much nitrogen as they could utilize. The unused nitrogen would not be expected to provide any yield benefit and probably would be volatilized or contaminate groundwater supplies (Environmental Protection Agency: National Pesticide Survey: Project Summary. US-EPA, Washington, D.C. (1990), which is hereby incorporated by reference in its entirety). No increase in yield of either silage or grain occurred above 150 lbs/ha of nitrogen in the presence of T-22. In the absence of T-22, however, the 150 lbs nitrogen was required for maximum yields as shown in FIGS. 6E-F, i.e., maximum yields were obtained with 53% less nitrogen in the presence of T-22. In order to comply with a 1998 federal law requiring a plan for controlling hypoxia in the Gulf of Mexico, (P.L. 105-383 Nov. 13, 1998), the US Environmental Protection Agency may mandate a reduction in nitrogen fertilizer use in the Mississippi River basin. A microbial agent that increases nitrogen use efficiency by crop plants would be useful in this regard.

A similar trial was established wherein soil types and planting conditions were quite similar but there was a high level of residual nitrogen in the field. PSNT values ranged from 40 to 90 ppm; corn usually does not respond to nitrogen fertilizer if values are above 20-30 ppm (Heckman et al., “Corn Response To Sidedress Nitrogen In Relation To Soil Nitrate Analysis,” Commun. Soil Sci. Plant Anal. 27:575-583 (1996), which is hereby incorporated by reference in its entirety). Under these conditions, there was no measurable response of the corn to application of T-22. However, roots were colonized by the fungus and deep root growth was enhanced; the data in FIGS. 5A-C are from this plot. Even in the presence of optimal or supraoptimal levels of nitrogen, T-22 apparently promotes increased corn deep root development, but unless nitrogen is limiting during some phase of the growth cycle of the corn, this deeper root system does not affect plant growth or yield in the absence of other stress factors such as drought.

More than 70 separate T-22 trials have been done on field corn alone, more than 30 on soybeans, and numerous trials have been done on crops such as wheat, peas, sugar beets and potatoes. Specific advantages and uses of T-22 on these crops are numerous and diverse. When T-22 is applied as a seed treatment on potatoes, both size (increased grade) and yields frequently are increased. When T-22 or T. virens strain 41 were applied in Brazil to wheat seeds infected with Pyrenophora tritici-repentis, both stands and yields were increased significantly and substantially (yields increased from 1,666 to 2,166 kg/ha for strain T-22, and to 1,953 kg/ha for strain 41) (da Luz et al., “Seed-Applied Bioprotectants For Control of Seedborne Pyrenophora fritici-repentis and Agronomic Enhancement of Wheat,” Can. J. Plant Pathol. 19:384-386 (1998), which is hereby incorporated by reference in its entirety). Similar results were obtained over 2 years with corn seeds infected primarily with Fusarium graminearum and F. moniliforme. Seed treatment with T-22 enhanced spring green-up and significantly reduced white heads caused by Gaeumannomyces graminis var. tritici from 32% to 21%.

### Example 9

### Tolerance to Biotic and Abiotic Stresses Through Enhanced Root Development

Another possible mechanism recently gaining credence is tolerance to stress through enhanced deep rooting and plant development. The drought tolerance and enhanced nitrogen utilization indicated in FIGS. 5A-C and FIGS. 6A-F are examples of this mechanism. The enhanced rooting by T-22 probably also induces tolerance to pests that it does not directly control. For example, it is thought that T-22 has no ability to control Phylophthora spp. because it has no mechanism to intercept or attack zoospores. Indeed, in several studies, it was found to have no effect on this pathogen (Smith et al., “Potential For Biological Control Of Phytophthora Root And Crown Rots of Apple By Trichoderma and Gliocladium spp.,” Phytopathology 80:880-885 (1990), which is hereby incorporated by reference in its entirety). However, growers have indicated that Phytophthora-attacked plants were larger in the presence of T-22 than in its absence. One possible explanation for this result is that the larger root systems of plants colonized by T-22 were better able to withstand the damaging effects of the pathogen.

### Example 10

### Deep Rooting Agents May Be Used in Combination with Other Treatments

In nearly all cases, the best results in direct-seeded field crops are obtained with an integration of chemical seed treatments and the deep root developing agents. Extensive testing has revealed almost no chemical seed treatments that prevent subsequent root colonization, for example, by T. harzianum strain T-22 (full rates of thiram and tebuconazole are exceptions). This resistance permits the use of both the biological root colonizing agent and chemical seed protectants.

Granules of T-22 frequently are incorporated into potting mixes in greenhouse crops for production of vegetable and flower transplants and also pot crops such as chrysanthemums and poinsettias. For example, tomatoes were grown in a potting mix containing the granular formulation of T-22 (BioWorks, Inc., Geneva, N.Y.) which permitted roots to become colonized, then transplanted to the field. Fusarium crown and root rot at harvest of mature fruit was reduced (Datnoff et al, “Biological Control Of Fusarium Crown And Root Rot Of Tomato In Florida Using Trichoderma harzianum and Glomus intraradices,” Biol. Contr. 5:427-431. (1995); Nemec et al., “Efficacy Of Biocontrol Agents In Planting Mixes To Colonize Plant Roots And Control Root Diseases Of Vegetables And Citrus,” Crop Protect. 15:735-742 (1996), which are hereby incorporated by reference in their entirety). T-22 was not the only effective organism; the rhizosphere competent Bacillus subtilis (the active ingredient in Kodiak) from Gustafson (Plano, Tex.) was also effective. The combination of T-22 and the mycorrhizal fungus Glomus intraradices was more effective than either organism alone (Datnoff et al, “Biological Control Of Fusarium Crown And Root Rot of Tomato In Florida Using Trichoderma harzianum and Glomus intraradices,” Biol. Contr. 5:427-431. (1995), which is hereby incorporated by reference in its entirety). Thus, application of a very small amount of any of several biocontrol organisms at the time of seeding of transplants provided a season-long benefit to tomato health and yield. Again, this is hardly a localized or transitory effect or one confined to seeds, seedlings or the infection court to which the biocontrol agent was originally applied.

There are similar studies on other transplant vegetables. Pepper seedlings were produced in the greenhouse with or without the addition of T-22. The peppers were transplanted into the field under less than ideal conditions. When their roots lacked T-22, fewer pepper plants survived transplanting and early yields were lower.

A number of trials have focused on ornamentals; generally, T-22 either applied as a drench or incorporated as granules gives adequate disease control over an extended period of time (several months). Further, root development frequently is better in plants where disease is controlled by T-22 instead of fungicides; many fungicides inhibit root growth to some extent while T-22 enhances deeper and more dense root development.

These data clearly demonstrate that T-22, as well as other rhizosphere competent biocontrol agents, can provide long-term protection or other advantages to plants from a single application at the beginning of the season. These biocontrol agents can establish themselves on roots, grow with the developing root system and remain functional for at least the life of an annual crop. Thus, biologicals can be more effective than chemical pesticides for root protection and plant growth enhancement. However, the assessment of efficacy is dependent upon the particular parameter being measured.

Expectations of the abilities of biocontrol agents are partially conditioned by the expectations of their mechanisms of action. If it is assumed that a specific agent (or group of agents such as Trichoderma spp.) has a single or a very limited number of mechanisms of action, it could be concluded that its activity might be specific to particular crops or pathogens. However, if it is determined that there are many different biochemicals, genes and even general modes of action for a specific BCA, then it is much more reasonable that the particular organism might have manifold and diverse advantages.

Further, the general conceptual framework in which any particular organism is viewed also affects one's thinking about its potential uses. Trichoderma spp. are frequently very numerous and even dominant in agricultural soils because they persist and multiply in the presence of healthy plant roots. In the absence of healthy roots, their numbers are likely to decline. Certainly this is true of T-22. If it is generally true of T. harzianum, these fungi, and others that are functionally similar, need to be considered as opportunistic plant root colonists or even symbionts. If so, then it is reasonable to assume that they will have developed numerous mechanisms to promote their ecological niche, i.e., abundant and healthy plant roots, which in turn provides multiple benefits to the plant.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of promoting plant deep root development, said method comprising:
applying Trichoderma spp. to a plant or plant seed under conditions effective to achieve deeper roots in the soil in a treated plant or plant grown from a treated seed than in untreated plants or plants grown from seed not treated with Trichoderma spp.

2. The method according to claim 1, wherein the Trichoderma spp. is Trichoderma harzianum.

3. The method according to claim 2, wherein T. harzianum is the protoplast fusion progeny T-22.

4. A method of reducing usage of nitrogen fertilizer in treating a plant, said method comprising:
applying a plant deep root developing agent to a plant or plant seed under conditions effective to reduce nitrogen fertilizer treatment of the plant while achieving a level of plant growth like that achieved when treating the plant with the nitrogen fertilizer but not the deep root developing agent.

5. A method according to claim 4, wherein the deep root developing agent is a plant enhancing microorganism or humate.

6. A method according to claim 5, wherein the deep root developing agent is a species of Trichoderma, Pseudomonas, Streptomyces, Bacillus, Burkholderia, or Fusarium, humic acid, or mixtures thereof.

7. The method according to claim 6, wherein the deep root developing agent is a species of Trichoderma.

8. The method according to claim 7, wherein the Trichoderma species is the protoplast fusion progeny T. harzianum T-22.

9. A method of imparting drought resistance to plants, said method comprising:
applying a plant deep root developing agent to a plant or plant seed under conditions effective to impart drought resistance to the plant or a plant grown from the plant seed.

10. A method according to claim 9, wherein the deep root developing agent is a plant enhancing microorganism or humate.

11. A method according to claim 10, wherein the deep root developing agent is a species of Trichoderma, Pseudomonas, Streptomyces, Bacillus, Burkholderia, or Fusarium, humic acid, or mixtures thereof.

12. The method according to claim 11, wherein the deep root developing agent is a species of Trichoderma.

13. The method according to claim 12, wherein Trichoderma is the protoplast fusion progeny T. harzianum T-22.

14. A method of increasing tolerance of plants to adverse soil conditions, said method comprising:
applying a plant deep root developing agent to a plant or plant seed under conditions effective to impart resistance to adverse soil conditions of the plant while achieving an improved level of plant growth.

15. A method according to claim 14, wherein the deep root developing agent is a plant enhancing microorganism or humate.

16. A method according to claim 15, wherein the deep root developing agent is a species of Trichoderma or humic acid, or mixtures thereof.

17. The method according to claim 16, wherein the deep root developing agent is a species of Trichoderma.

18. The method according to claim 17, wherein the Trichoderma species is the protoplast fusion progeny T. harzianum T-22.

19. A method according to claim 14, wherein the adverse soil condition is soil compaction.
